(19) United States
(12) Patent Application Publication
(10) Pub. No.: US 2016/0193419 A1
(43) Pub. Date: Jul. 7, 2016

(54) Drug Delivery Device

(71) Applicant: SANOFI-AVENTIS DEUTSCHLAND GMBH, Frankfurt am Main (DE)

(72) Inventors: Philip OAKLAND, Cheadle Heath, Stockport (GB); Steven David BUTLER, Essington, South Staffordshire (GB); Mark Philip HORLOCK, Timperley, Cheshire (GB)

(21) Appl. No.: 14/909,249

(22) PCT Filed: Aug. 5, 2014
(86) PCT No.: PCT/EP2014/066769

§ 371 Date: Feb. 1, 2016

(30) Foreign Application Priority Data
Aug. 6, 2013 (EP) 13179346.5

(51) Int. Cl. A61M 5/315

(52) U.S. Cl. CPC A61M 5/31513

(57) ABSTRACT

A drug delivery device (1) is disclosed which comprises a housing (2) having a longitudinal axis, a piston rod (3), at least one resilient guide member (4) for guiding the piston rod (3) in a drug dispense operation and a cartridge holder (5) for receiving a cartridge (6), wherein the cartridge holder (5) is detachable from the housing (2). Furthermore the drug delivery device (1) is configured such that the resilient guide member (4) is allowed to relax when the cartridge holder (5) is detached from the housing (2).

The present invention relates to a drug delivery device.

WO 03/008023 A1 describes a reusable injector pen, wherein, after the quantity of medicine contained in a cartridge therein is exhausted by multiple operations of the pen, the cartridge can be removed and replaced by a full cartridge.

It is an object of the invention to provide a drug delivery device providing an improved reset function.

This object may be achieved by a drug delivery device according to the independent claim. Further features, advantages and expediencies are subject-matters of the depending claims.

According to one aspect, a drug delivery device comprises a housing having a longitudinal axis and a piston rod.

The drug delivery device may be an injection device, preferably a pen-type injection device. Preferably, the device is a reusable device such that a cartridge containing a medicament can be exchanged. The term “housing” shall preferably mean any exterior housing (“main housing”, “body”, “shell”) or interior housing (“insert”, “inner body”) which may have a unidirectional axial coupling to prevent proximal movement of specific components. The housing may be designed to enable the safe, correct, and comfortable handling of the medication delivery device or any of its mechanism. Usually, it is designed to house, fix, protect, guide, and/or engage with any of the inner components of the medication delivery device (e.g., the drive mechanism, cartridge, piston, piston rod), preferably by limiting the exposure to contaminants, such as liquid, dust, dirt etc. In general, the housing may be unitary or a multipart component of tubular or non-tubular shape.

The piston rod may have a start position relative to the housing and may be resettable to the start position. For example, the piston rod may be resettable after replacement of a cartridge retaining a medication. Preferably, thereby the drug delivery device is reset in order to dispense a first dose of medication from the cartridge.

Here, the term “piston rod” is used for a component of a drug delivery device which, by carrying out a movement towards a dispensing end of the drug delivery device, causes medicament to be dispensed from the device. In particular, the piston rod may act on a piston or bung in a medicament container, for example a cartridge, causing medicament to be dispensed from the container. The piston rod may be configured for carrying out a combined axial and rotational movement. As an example, it may be a simple rod or a lead-screw having threads for engaging with corresponding parts of the drug delivery device. The piston rod may be of a unitary or a multipart construction.

According to one embodiment, the drug delivery device further comprises at least one resilient guide member for guiding the piston rod in a drug dispensing operation. The guide member is preferably fixed to the housing, so that, at least during the drug dispense operation, no relative movement between the guide member and the housing occurs.

According to an embodiment the piston rod is threadedly engageable with the guide member. The term “threadedly engageable” shall particularly mean the ability of an interlocking of one or more threads of components. The term “thread” shall preferably mean a full or part thread, for example a cylindrical spiral rib or groove located on the internal and/or external surface of a component of the drug delivery device. For example, the guide member may comprise or form a threaded nut threadedly engaged with the piston rod during a dose dispense operation and configured such that it allows the piston rod to advance forward in a distal direction towards the dispensing end of the drug delivery device, e.g. by rotating through the threaded nut. Furthermore, the drug delivery device may comprise a drive member acting on the piston rod and driving the piston rod in a dose dispense operation in a distal direction of the device.

Preferably, the drug delivery device further comprises a cartridge holder for receiving a cartridge. In particular, the cartridge holder may be detachable from the housing and may be configured to be re-attached to the housing after detachment. Advantageously, this allows a replacement of the cartridge arranged in the cartridge holder, e.g. in order to replace an empty cartridge by a full cartridge.

According to an embodiment, the drug delivery device is configured such that the resilient guide member is allowed to relax when the cartridge holder is detached from the housing.

For example, the guide member may comprise a “relaxed” position and may be moved out or displaced from its “relaxed” position by application of a force acting on the guide member. When such force stops acting on the resilient guide member, the guide member returns to the “relaxed” position due to its resilient force. Such force forcing the guide member to leave its “relaxed” position may for example be applied to the guide member by the cartridge holder when the cartridge holder is attached to the housing of the device. Advantageously, the guide member is moved into engagement with the piston rod, when the cartridge holder is attached to the housing. In particular, the guide member may be resiliently deformed to engage with the piston rod. Advantageously, a disengagement of the guide member and the piston rod may occur when the cartridge holder is detached from the housing.

According to an embodiment, the guide member forms an integral part of the housing. In particular, the guide member may comprise one or more elements that project, for example from an outer housing part, into the interior of the housing.

According to an embodiment, the piston rod comprises a threaded section. The piston rod may comprise one or a plurality of threaded sections. Preferably, the piston rod comprises a lead-screw with an external thread. Preferably, the lead-screw is threadedly engaged with the guide member during a dose dispense operation of the drug delivery device.

According to an embodiment, the guide member comprises at least one finger configured for engagement with the piston rod. In particular, the finger may comprise a portion of thread suited for engaging with one or more threaded sections of the piston rod.

According to an embodiment, the guide member comprises a plurality of fingers threadedly engageable with the piston rod. For example, the guide member may comprise two, three or more fingers that are configured to engage with the piston rod when the cartridge holder is attached to the housing. Preferably, each of the fingers comprises at least one portion of thread that may engage with one ore more threaded sections of the piston rod. Advantageously, the fingers may engage with the piston rod at different sides of the piston rod resulting in a particular reliable guidance of the piston rod.

According to an embodiment, the cartridge holder directly interacts with the guide member when the cartridge holder is attached to the housing. For example, the cartridge holder may directly push on the guide member or on parts of the guide member, thereby moving the guide member or parts of it to a position different from its relaxed position when the cartridge holder is detached, in particular to a position wherein the guide member is engaged with the piston rod.

According to an embodiment, the drug delivery device is configured such that the guide member moves out of engagement with the piston rod when the cartridge holder is detached from the housing. Preferably, the guide member moves back to its relaxed position it had assumed when the cartridge holder was not yet attached.

According to an embodiment, the cartridge holder comprises at least one angled face configured for a mechanical interaction with the guide member. For example, the one ore more angled faces of the cartridge holder may be configured for pushing the guide member and/or its fingers towards the piston rod. Preferably, the guide member comprises at least one angled face configured for a mechanical interaction with the at least one angled face of the cartridge holder. Preferably, the shape and size of the angled faces of the cartridge holder and the guide member are formed such that they can easily interact with each other. Furthermore, the angled faces may at least partially transform a force acting in an axial direction into a force acting in a radial direction.

According to an embodiment, the drug delivery device is configured such that the piston rod is enabled to be moved back in a proximal direction towards a start position after detachment of the cartridge holder. For example, the piston rod may be pushed back or wound back in the proximal direction. Hence, the piston rod can be reset to its start position unhindered by a thread of the guide member or a threaded nut formed by the guide member. Advantageously, the drug delivery device is configured such that a very light reset force is needed for pushing back the piston rod to its start position, since the piston rod becomes a disengaged body in regard to the guide member. Therefore, forces required in order to reset the device may be significantly low.

According to an embodiment, the drug delivery device is configured such that the guide member is pushed towards the piston rod against its resilient force when the cartridge holder is attached to the housing. Preferably, the cartridge holder, for example the angled faces of the cartridge holder, may push the guide member, in particular the one or more fingers of the guide member, towards the piston rod, forcing the guide member and/or the fingers to leave their “relaxed” position. Thereby, the one or more fingers of the guide member may engage with the piston rod, for example by interlocking of the threads of the fingers with the threaded sections of the piston rod.

Advantageously, the drug delivery device is characterized in that it can be used very intuitively. In addition, very few steps are required in order to exchange the cartridge of the device. In particular, after detaching the cartridge holder from the housing in order to replace the cartridge of the device, a user may simply need to move back the piston rod and re-attach the cartridge holder containing the new cartridge, without having to take any additional action regarding the disengagement of the piston rod from the guide member. By re-attaching the cartridge holder to the housing of the device, the guide member may automatically engage with the piston rod, again without requiring any additional actions of the user of the device. Furthermore, preferably, the resetting of the drug delivery device by pushing back the piston rod after disengagement from the guide member due to the detachment of the cartridge holder may not disturb any other internal parts of the drug delivery device or its mechanism.

The term “medication” or “drug”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,
wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,
wherein in a further embodiment the pharmaceutically active compound comprises at least one peptide for the treatment and/or prophylaxis of diabetes mellitus or complications associated with diabetes mellitus such as diabetic retinopathy,
wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and insulin and B29-N-(ω-carboxyheptadecanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly- Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 Exendin-4(1-39),

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(02)25, Asp28] Exendin-4(1-39),

des Pro36 [Met(O)14 Trp(02)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(02)25, Asp28] Exendin-4(1-39),

des Pro36 [Met(O)14 Trp(02)25, IsoAsp28] Exendin-4(1-39),

wherein the group -Lys6-NH2 may be bound to the C-terminus of the Exendin-4 derivative;

or an Exendin-4 derivative of the sequence

des Pro36 Exendin-4(1-39)-Lys6-NH2 (AVE0010),

H-(Lys)6-des Pro36 [Asp28] Exendin-4(1-39)-Lysb-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(02)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; a and y contain approximately 450 amino acids and δ approximately 500 amino acids, while μ and ε have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H-H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

Further features, refinements and expediencies become apparent from the following description of the exemplary embodiments in connection with the figures.

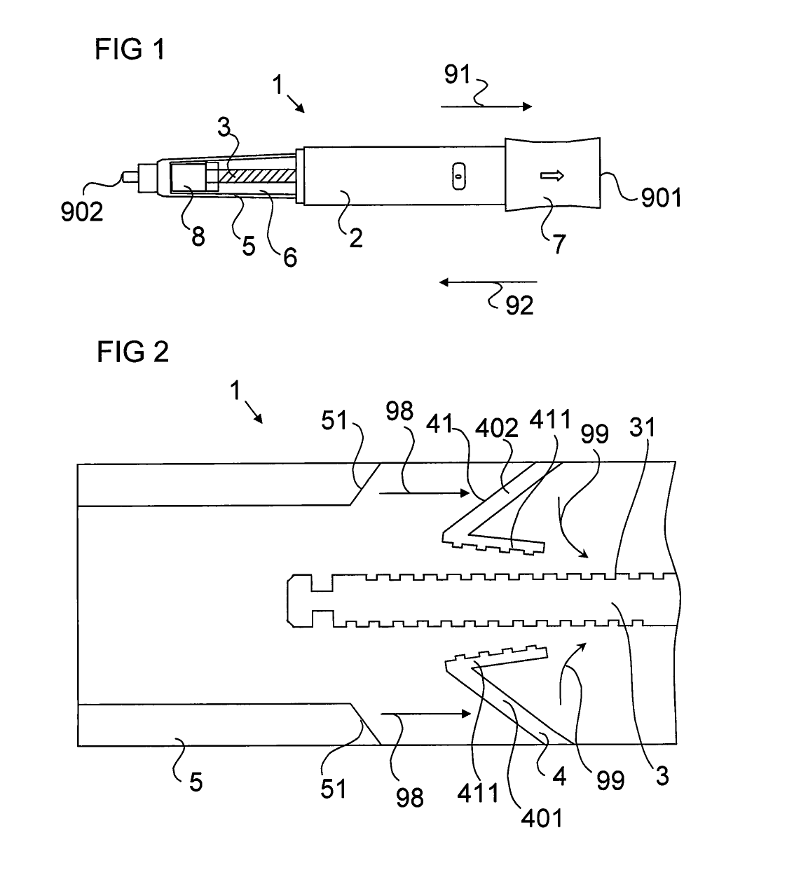

FIG. 1 schematically shows a perspective side view of a drug delivery device.

FIG. 2 schematically shows a sectional side view of a part of the drug delivery device of FIG. 1.

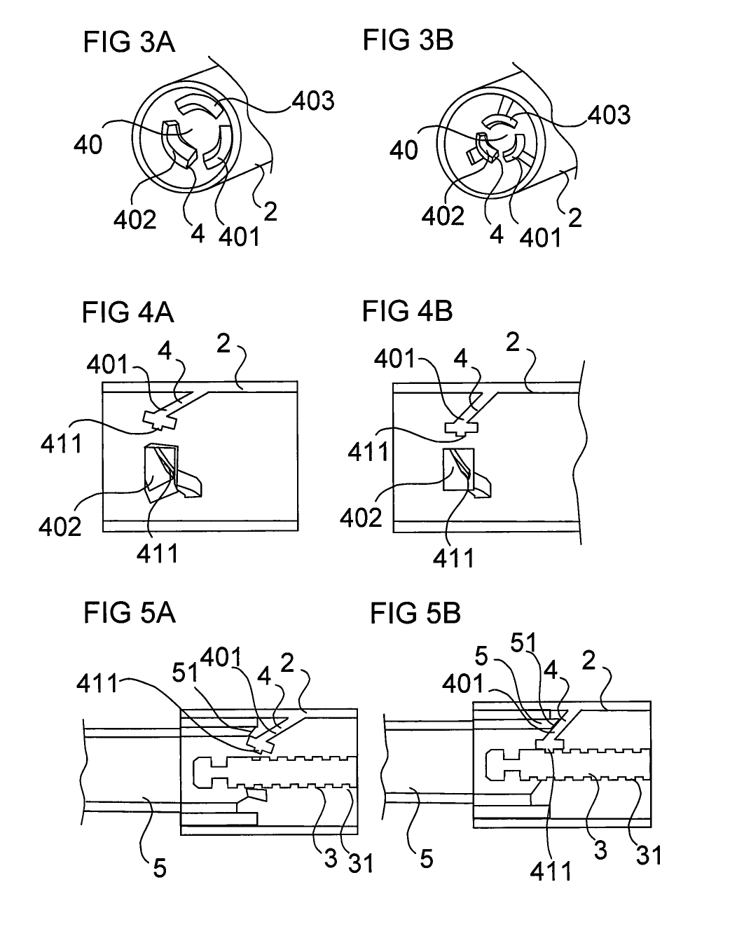

FIGS. 3A and 3B schematically show a perspective view of a part of the housing of a drug delivery device.

FIGS. 4A to 5B schematically show sectional side views of a resilient guide member.

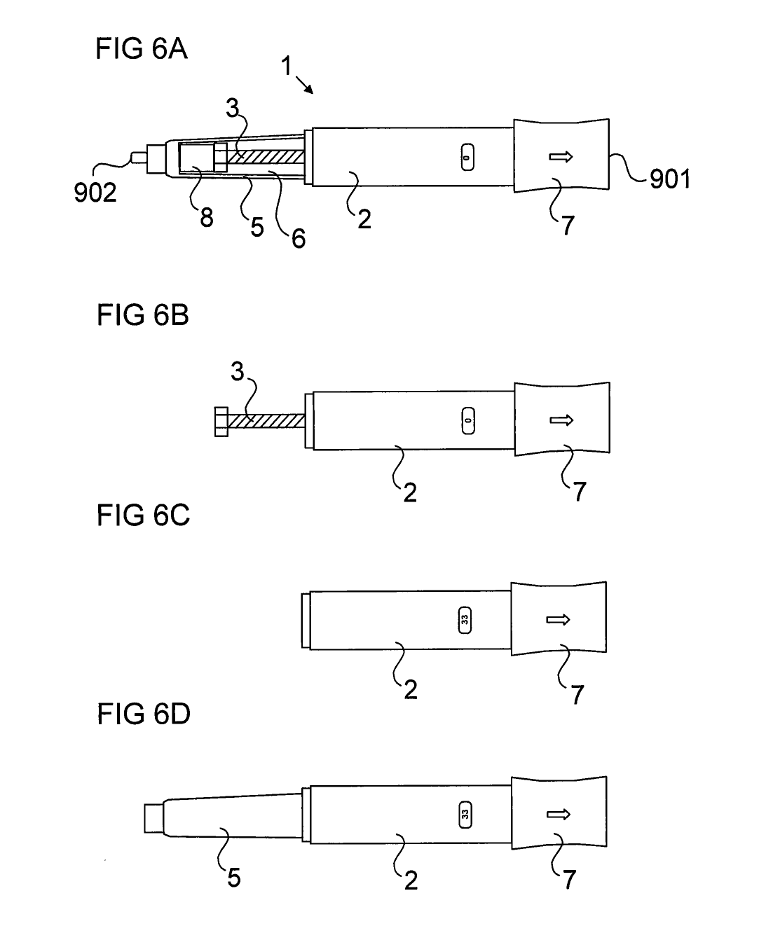

FIGS. 6A to 6D schematically show perspective side views of the drug delivery device of FIG. 1 at different stages of the resetting of the device.

Like elements, elements of the same kind and identically acting elements may be provided with the same reference numerals in the figures.

FIG. 1 shows an embodiment of a drug delivery device 1 which comprises a housing 2 and a cartridge holder 5 that is attached to the housing 2. Preferably the cartridge holder 5 is suited for holding a cartridge 6 which may contain a medication. The housing 2, the cartridge holder 5 and/or the cartridge 6 may have a tubular shape. The drug delivery device 1 comprises a dose button 7 for setting and dispensing a dose of a medication. The drug delivery device 1 may be a pull-push device such that the dose button 7 is pulled out of the housing 2 for setting a dose and is pushed towards the housing 2 for dispensing a dose.

The medication retained in the cartridge 6 is preferably liquid medication. The cartridge 6 preferably comprises a plurality of doses of the medication. The medication may comprise insulin, heparin, or growth hormones, for example. The cartridge 6 has an outlet at its distal end. Medication can be dispensed from the cartridge through its outlet. The device 1 may be a pen-type device, in particular a pen-type injector. The device 1 may be reusable device. The device 1 may be a device configured to dispense fixed doses of the medication or variable, preferably user-settable, doses. The device 1 may be a needle-based or a needle free device. The device 1 may be an injection device.

The term “distal end” of the medication delivery device 1 or a component thereof may refer to that end of the device or the component which is closest to the dispensing end of the device 1. The term “proximal end” of the medication delivery device 1 or a component thereof may refer to that end of the device or the component which is furthest away from the dispensing end of the device. In FIG. 1, the distal end of the device 1 is assigned reference numeral 902 and the proximal end of the device is assigned reference numeral 901. The arrows 91 and 92 are used to indicate the proximal and the distal direction, i.e. me direction towards me proximal end 901 and towards the distal end 902.

A piston 8 is retained within the cartridge 6. The piston 8 is movable with respect to the cartridge 6. The piston 8 may seal the medication within the cartridge 6. The piston 8 expediently seals the interior of the cartridge 6 proximally. Movement of the piston 8 with respect to the cartridge 6 in the distal direction causes medication to be dispensed from the cartridge 6 through its outlet during operation of the device 1.

The drug delivery device 1 further comprises a piston rod 3. The piston rod 3 may be configured for transferring force to the piston 8, thereby displacing the piston in the distal direction 92 with respect to the cartridge 6. A distal end face of the piston rod 3 may be arranged to abut a proximal end face of the piston 8.

The drug delivery device 1 may comprise a drive member (not shown in this figure) for transferring a force from the dose button 7 to the piston rod 3 and, thereby, causing a displacement of the piston rod 3 in a dose dispense operation. Furthermore, the drug delivery device 1 may comprise a guide member (not shown in this figure) for guiding the piston rod 3 in a dose dispense operation.

The piston rod 3 may be flexible or not. It may be a simple rod, a lead-screw, a rack and pinion system, a worm gear system, or the like. It may have a circular or non-circular cross-section. It may be made of any suitable material known by a person skilled in the art and may be of unitary or multipart construction.

The drug delivery device 1 may be a manually, in particular non-electrically, driven device.

FIG. 2 schematically shows a sectional side view of a part of the drug delivery device 1 of FIG. 1 in a state where the cartridge holder 5 is detached from the housing 2. The housing 2 comprises a resilient guide member 4 comprising two flexible fingers 401, 402 that are configured for engagement with the piston rod 3 in dose set and dispense operations of the device 1. The flexible fingers 401, 402 are in a relaxed state, disengaged from the piston rod 3. Preferably, the guide member 4 and/or the fingers 401, 402 form an integral part of the housing 2. The piston rod 3 is a lead-screw comprising a threaded section 31. In a dose dispense operation the lead-screw may advance forward by rotating through a thread 411 of the fingers 401, 402.

Furthermore, the cartridge holder 5 comprises angled faces 51 which are configured for engagement with angled faces 41 of the fingers 401, 402 of the resilient guide member 4. In FIG. 2, two arrows 98 pointing in the proximal direction of the drug delivery device are used to show that the cartridge holder 5 is being attached to the housing 2. During attachment of the cartridge holder 5, the angled faces 51 of the cartridge holder 5 push the fingers 401, 402 against their resilient force in a direction indicated by arrows 99 until the threads 411 of the fingers 401, 402 are forced into engagement with the threaded section 31 of the lead-screw. Thereby, the angled faces 51 may at least partially transform an axial force into a radial force.

For the replacement of medication cartridge 6, the cartridge holder 5 is detached from the housing 2, allowing the fingers 401, 402 of the resilient guide member 4 to relax. Consequently, the fingers 401, 402 move out of engagement with the lead-screw and, in particular, the threads 411 of the fingers 401, 402 move out of the threaded section 31 of the lead-screw. This allows the lead-screw to be moved back into the housing 2 unhindered by the thread of the threaded section 31. Thus, the drug delivery device can be reset in an effective manner, for example by a rotational and/or by a translational movement of the lead-screw towards its start position.

FIGS. 3A to 5B schematically show perspective and sectional side views of a part of the housing 2 of a drug delivery device 1 of FIGS. 1 and 2. FIGS. 3A and 3B show a perspective view of the distal end of the housing 2 such that the guide member 4 is visible. FIGS. 4A to 5B show sectional side views of a part of the housing 2 and of the guide member 4 located in the housing 2. For clarity reasons, the piston rod 3 is not depicted in FIGS. 3A, 3B, 4A and 4B. The resilient guide member 4 comprises three flexible fingers 401, 402, 403 that form an opening 40 configured such that the piston rod can move through the opening 40. The fingers 401, 402, 403 are configured to engage with one or more threaded sections 31 of the piston rod 3. Preferably, the fingers 401, 402, 403 comprise one or more portions of thread 411 that are configured to engage with the threaded section 31 of the piston rod 3 when the cartridge holder 5 is attached to the housing.

In FIGS. 3A, 4A and 5A, the cartridge holder 5 is detached from housing 2, allowing the fingers 401, 402, 403 to relax. As a consequence, the diameter of the opening 40 formed by the fingers 401, 402, 403 increases and the fingers 401, 402, 403 become disengaged from the piston rod 3, i.e. the portions of thread 411 of the fingers 401, 402, 403 are out of engagement from the threaded section 31 of the piston rod 3. Thus, the piston rod 3 can be pushed back or wound back in the proximal direction 91 for a resetting of the drug delivery device 1.

In FIGS. 3B, 4B and 5B, the cartridge holder 5 is attached to the housing 2 (the cartridge holder 5 is not shown in FIG. 3B and 4B). As a consequence of the attachment of the cartridge holder 5, the flexible fingers 401, 402, 403 elastically deform by pivoting down onto the piston rod 3, thereby forming a threaded nut engaging with the threaded section 31 of the piston rod. In particular, when attaching the cartridge holder 5 to the housing 2, the cartridge holder 5 presses the guide member 4 in the proximal direction against its resilient force until the threads 411 of the fingers 401, 402, 403 of the guide member 4 engage with the threaded section 31 of the piston rod 3. As a consequence, the diameter of the opening 40 formed by the fingers 401, 402, 403 is decreased so that the guide member 4 may function as a threaded nut for the piston rod 3.

FIGS. 6A to 6D schematically show perspective side views of the drug delivery device 1 at different stages of the resetting of the device 1 of FIG. 1.

In FIG. 6A, the cartridge holder 5 is attached to the housing 2 of the drug delivery device 1. As described in connection with the FIGS. 2 to 5B, the guide member 4 forms a threaded nut for the piston rod 3, thereby guiding the piston rod 3 in a dose dispense operation.

FIG. 6B shows the device 1 after detachment of the cartridge holder 5. The fingers 401, 402, 403 of the guide member 4 are relaxed and disengaged from the piston rod as shown in FIGS. 3A, 4A and 5A. In this state, the piston rod 3 is enabled to be pushed back in the proximal direction 91 back to its start position unhindered by the guide member 4.

FIG. 6C shows the drug delivery device 1 after the piston rod 3 has been pushed back to its start position.

Finally, as shown in FIG. 6D, the cartridge 6 can be replaced and the cartridge holder 5 can be re-attached to the housing 2, thereby engaging the guide member 4 with the piston rod 3. In this state, the device 1 is ready for new dose set and dispense operations.

The invention is not restricted by the description on the basis of the exemplary embodiments. Rather, the invention encompasses any new feature and also any combination of features, which in particular comprises any combination of features in the patent claims, even if this feature or this combination itself is not explicitly specified in the patent claims or exemplary embodiments.

### REFERENCE NUMERALS

1 drug delivery device

2 housing

3 piston rod

31 threaded section

4 guide member

40 opening

401, 402, 403 finger

411 thread

5 cartridge holder

41, 51 angled face

6 cartridge

7 dose button

8 piston

91 proximal direction

92 distal direction

901 proximal end

902 distal end

98, 99 arrows

1. A drug delivery device (1) comprising:
a housing (2) having a longitudinal axis,
a piston rod (3),
at least one resilient guide member (4) for guiding the piston rod (3) in a drug dispense operation,
a cartridge holder (5) for receiving a cartridge (6),
wherein the cartridge holder (5) is detachable from the housing (2), and
wherein the drug delivery device (1) is configured such that the resilient guide member (4) is allowed to relax when the cartridge holder (5) is detached from the housing (2).

2. The drug delivery device according to claim 1, wherein the guide member (4) forms an integral part of the housing (2).

3. The drug delivery device according to claim 1 or 2, wherein the piston rod (3) comprises a threaded section (31).

4. The drug delivery device according to one of the proceeding claims, wherein the guide member (4) comprises at least one finger (401) configured for engagement with the piston rod (3).

5. The drug delivery device according to claim 4, wherein the at least one finger (401) comprises a portion of a thread (411).

6. The drug delivery device according to one of the proceeding claims, wherein the guide member (4) comprises a plurality of fingers (401, 402, 403) threadedly engageable with the piston rod (3).

7. The drug delivery device according to one of the proceeding claims, wherein the cartridge holder (5) directly interacts with the guide member (4) when the cartridge holder (5) is attached to the housing (2).

8. The drug delivery device according to one of the proceeding claims, wherein the piston rod (3) is threadedly engageable with the guide member (4).

9. The drug delivery device according to one of the proceeding claims, configured such that the guide member (4) moves out of engagement with the piston rod (3) when the cartridge holder (5) is detached from the housing (2).

10. The drug delivery device according to one of the proceeding claims, wherein the cartridge holder (5) comprises at least one angled face (51) configured for a mechanical interaction with the guide member (4).

11. The drug delivery device according to claim 10, wherein the guide member (4) comprises at least one angled face (41) configured for a mechanical interaction with the at least one angled face (51) of the cartridge holder (5).

12. The drug delivery device according to one of the proceeding claims, configured such that the piston rod (3) is enabled to be moved back in a proximal direction (91) towards a start position (81) after detachment of the cartridge holder (5).

13. The drug delivery device according to one of the proceeding claims, configured such that the guide member (4) is pushed towards the piston rod (3) contrary its resilient force when the cartridge holder (5) is attached to the housing (2).
